(19) United States
(12) Patent Application Publication
(10) Pub. No.: US 2026/0224116 A1
(43) Pub. Date: Aug. 6, 2026

(54) BIOLOGICAL INFORMATION MEASUREMENT DEVICE, CONTROL METHOD OF BIOLOGICAL INFORMATION MEASUREMENT DEVICE, INFORMATION PROCESSING SYSTEM, AND STORAGE MEDIUM

(71) Applicant: OMRON HEALTHCARE CO., Ltd., Muko-shi (JP)

(72) Inventors: Kentaro MORI, Kyoto (JP); Tsuyoshi Kitagawa, Kyoto (JP); Hiroko Yoshino, Kyoto (JP); Sogo Nakashima, Kyoto (JP); Kazuyuki Nakagawa, Kyoto (JP)

(73) Assignee: OMRON HEALTHCARE Co. Ltd., Muko-shi (JP)

(21) Appl. No.: 19/445,857

(22) Filed: Jan. 12, 2026

(30) Foreign Application Priority Data
Feb. 5, 2025 (JP) 2025-017921

(51) Int. Cl. A61B 5/0205; A61B 5/00; A61B 5/021; A61B 5/11; A61B 5/361

(52) U.S. Cl. CPC A61B 5/0205; A61B 5/02108; A61B 5/11; A61B 5/361; A61B 5/742; A61B 5/7475

(57) ABSTRACT

A biological information measurement device including: a blood pressure measurement unit that acquires information for measuring blood pressure; an arrhythmia information acquisition unit that acquires information related to presence or absence of arrhythmia in the living body during measurement of the blood pressure; a display unit including an area capable of displaying the blood pressure; an input unit that receives an operation input by a user; and a control unit, wherein the control unit calculates the blood pressure on the basis of the information acquired by the blood pressure measurement unit, determines presence or absence of arrhythmia during the measurement of the blood pressure, and causes, in a case where arrhythmia has been present during the measurement of the blood pressure, the display unit not to display a value of the blood pressure measured but to display information of a fact that arrhythmia has been present.

### CROSS-REFERENCE TO RELATED APPLICATIONS

The disclosure of Japanese Patent Application No.JP 2025-017921, filed on February 5, 2025 including the specification, drawings and abstract is incorporated herein by reference in its entirety.

### TECHNICAL FIELD

The present invention relates to a biological information measurement device capable of measuring blood pressure of a living body, a control method of a biological information measurement device, an information processing system, and a program.

### BACKGROUND ART

In recent years, it has become widespread to perform health management by measuring information regarding the body and health of an individual such as a blood pressure value with a measuring instrument and recording and analyzing the measurement result. In particular, since arrhythmia such as atrial fibrillation (AF) may lead to a brain/cardiovascular disease, it is desirable that the user himself/herself can easily recognize such an abnormality.

In this regard, for example, there has been proposed a sphygmomanometer that displays, in a case where atrial fibrillation has been detected at the time of blood pressure measurement, the fact that there has been atrial fibrillation together with a blood pressure measurement result (for example, Patent Document 1). According to such a sphygmomanometer, the user can quickly recognize the presence of arrhythmia while performing daily health management of blood pressure measurement.

Furthermore, Patent Document 2 that discloses a biological information acquisition device discloses that, in a case where arrhythmia has been detected in an electrocardiogram test performed prior to blood pressure measurement, notifying the detection of arrhythmia makes it possible to notify the user of a decrease in reliability of the blood pressure measurement.

### PRIOR ART DOCUMENTS

### PATENT DOCUMENTS

### Patent Document 1: JP 2024-039189 A

### Patent Document 2: JP 2008-168074 A

### SUMMARY

However, if the blood pressure value is displayed as the measurement result even in a case where arrhythmia occurs, the user may focus on the displayed blood pressure value and overlook the occurrence of the arrhythmia. In the blood pressure measurement result when arrhythmia has been detected, the blood pressure value cannot have been accurately measured and is inaccurate. Therefore, the user erroneously recognizes the inaccurate blood pressure value as a result of appropriately measuring the blood pressure value in a case where the information regarding the occurrence of arrhythmia is overlooked.

In view of the above circumstances, an object of the present invention is to provide a technology capable of preventing an inaccurate blood pressure measurement result from being indicated to a user and of notifying the user of the arrhythmia detected during blood pressure measurement.

In order to solve the above problem, the present invention adopts the following configuration. The configuration is

a biological information measurement device including:

a blood pressure measurement unit that acquires information for measuring blood pressure of a living body;

an arrhythmia information acquisition unit that acquires information related to presence or absence of arrhythmia in the living body during measurement of the blood pressure;

a display unit including an area capable of displaying at least the blood pressure;

an input unit that receives an operation input by a user; and

a control unit, wherein

the control unit

calculates the blood pressure on the basis of the information acquired by the blood pressure measurement unit, and

causes, in a case where the information acquired by the arrhythmia information acquisition unit indicates that arrhythmia during the measurement of the blood pressure has been present, the display unit not to display a value of the blood pressure measured but to display information of a fact that arrhythmia has been present.

Note that the information related to the presence or absence of arrhythmia acquired by the arrhythmia information acquisition unit may be information on the presence or absence of arrhythmia detected by another biological information measurement device or a biological signal itself measured by another device. Examples of “arrhythmia” include atrial fibrillation, atrial flutter, and premature contraction.

According to such a configuration, in a case where arrhythmia has been present during the blood pressure measurement, only the fact that arrhythmia has been present is displayed without displaying the blood pressure value. Therefore, it is possible to prevent the inaccurate blood pressure measurement result from being indicated to the user and to notify the user of the arrhythmia detected during the blood pressure measurement.

### Furthermore, the control unit

may calculate a first average value that is an average value of the blood pressure of a case where the measurement of the blood pressure is performed a plurality of times at predetermined intervals according to an operation by a user, and causes the display unit to display the first average value, and

causes, in a case where the information acquired by the arrhythmia information acquisition unit indicates that arrhythmia has been present during the measurement in any of the plurality of times of measurement of the blood pressure, the display unit not to display the first average value but to display information of the fact that arrhythmia has been present.

For example, in a case where blood pressure is measured in the morning and the night, it is desirable from the viewpoint of health management that blood pressure is measured a plurality of times at short intervals and an average value thereof is used as a blood pressure value in one measurement opportunity, because the measurement values show less variation as compared with a case where measurement is performed only once in each measurement opportunity. According to the configuration as described above, even in a case where the measurement value of one measurement opportunity is provided using the average value of measurement values from such plurality of times of measurement, it is possible to prevent an inaccurate blood pressure measurement result from being indicated to the user and to promptly notify the user of arrhythmia.

In the present specification, terms such as first and second are used to describe various components, but the terms are used only for the purpose of distinguishing one component from other components, and do not mean any hierarchy or order. For example, the first component can be referred to as a second component without departing from the scope of rights of the present invention, and the second component can also be referred to as a first component.

The biological information measurement device may further include a storage unit that holds information related to measurement of the blood pressure performed in the past, the information including information related to a date and time of the measurement and presence or absence of arrhythmia during the measurement at the date and time, and

the control unit

may receive, via an input unit, a request to display, on the display unit, the information related to the measurement performed in a past of the blood pressure, the information being held in the storage unit, and

may cause, in a case where the information related to the measurement of the blood pressure related to the request to display includes information of the fact that arrhythmia has been present during the measurement, the display unit not to display a value of the blood pressure at the time of the measurement performed in a past but to display the information of the fact that arrhythmia has been present.

According to such a configuration, the user can view the past measurement history, and even in a case where arrhythmia has been detected at the time of the past measurement, it is possible to prevent an inaccurate blood pressure measurement result from being indicated to the user, and to notify the user of the arrhythmia detected during the blood pressure measurement.

Furthermore, the control unit

may receive, via the input unit, a request to calculate a second average value that is an average value of the blood pressure for each measurement time zone in past any period using the information related to the measurement of the blood pressure held in the storage unit,

may calculate, in a case where any of a plurality of pieces of the information related to the measurement of the blood pressure related to the request to calculate includes information of the fact that arrhythmia has been present during the measurement of the blood pressure, the second average value without using the information related to the measurement of the blood pressure, and

may cause the display unit to display the second average value calculated.

According to such a configuration, the user can view the average value for each time zone (for example, measurement opportunity at morning/night) in past any period (for example, last one week), and it is possible to prevent the average value from being calculated using the inaccurate measurement value of the case where arrhythmia has been detected.

Furthermore, the control unit may detect body motion during the measurement of the blood pressure on the basis of the information acquired by the blood pressure measurement unit, and may cause, in a case where the number of times of detection of the body motion during the measurement of the blood pressure is equal to or more than a predetermined value, the display unit to display that the number of times of detection of the body motion during the measurement of the blood pressure is equal to or more than the predetermined value. With such a configuration, in a case body motion during measurement is frequent, it is possible to indicate to the user the possibility that the accuracy of the measured blood pressure value has decreased.

Furthermore, the arrhythmia information acquisition unit may acquire the information related to presence or absence of arrhythmia in the living body on the basis of pulse wave information acquired by the blood pressure measurement unit. For example, in a case where the blood pressure measurement unit calculates the blood pressure on the basis of the pressure pulse wave, arrhythmia can be detected on the basis of the pressure pulse wave.

Furthermore, the biological information measurement device may further include an electrocardiographic waveform acquisition unit that acquires an electrocardiographic waveform of the living body, wherein the arrhythmia information acquisition unit may acquire the information related to presence or absence of arrhythmia in the living body on the basis of the electrocardiographic waveform during the measurement of the blood pressure.

Furthermore, the control unit may cause, in a case where a user operation to display a blood pressure value measured when arrhythmia has been present is received via the input unit after causing the display unit not to display the value of the blood pressure measured but to display information of the fact that arrhythmia has been present, the display unit to display the blood pressure value.

According to such a configuration, the user can recognize the occurrence of arrhythmia, and, based on the fact (that is, on the premise that the measurement value is inaccurate), can refer to the blood pressure value measured.

Furthermore, the present invention can also be regarded as a method as follows. The method is

a control method of a biological information measurement device, including:

measuring blood pressure of a living body;

determining presence or absence of arrhythmia in the living body during measurement of the blood pressure; and

displaying a measurement result of the blood pressure, wherein,

in a case where it is determined, in the determining presence or absence of arrhythmia, that arrhythmia has been present, a value of the blood pressure measured is not displayed but information of a fact that arrhythmia has been present is displayed in the displaying the measurement result.

Furthermore, the present invention can also be regarded as a program for causing the biological information measurement device to execute the above-described control method, and a computer-readable recording medium in which such a program is non-transiently recorded.

Furthermore, the present invention can also be regarded as a system as follows. The system is

an information processing system including: a biological information measurement device that measures at least blood pressure of a living body; and an information processing terminal communicatively connected to the biological information measurement device, wherein

the information processing terminal

receives, from the biological information measurement device, at least determination information for determining presence or absence of arrhythmia during measurement of the blood pressure of the living body,

determines presence or absence of arrhythmia during the measurement of the blood pressure of the living body on the basis of the determination information, and

transmits, in a case where it is determined that arrhythmia has been present during the measurement of the blood pressure of the living body, information of a fact that arrhythmia has been present during the measurement of the blood pressure of the living body to the biological information measurement device, and wherein

the biological information measurement device includes a first display unit, and

causes, in a case where the information of the fact that arrhythmia has been present during the measurement of the blood pressure of the living body is received from the information processing terminal, the first display unit not to display a value of the blood pressure measured but to display the information of the fact that arrhythmia has been present.

According to such a configuration, the calculation load of the biological information measurement device can be reduced, and even if a high-performance calculation device is not used, it is possible to prevent an inaccurate blood pressure measurement result from being indicated to the user and to notify the user of the arrhythmia detected during the blood pressure measurement.

Furthermore, the biological information measurement device may include an electrocardiographic waveform acquisition unit that acquires an electrocardiographic waveform of the living body, and the determination information may be the electrocardiographic waveform.

Furthermore, the information processing terminal may include a second display unit, may receive a blood pressure value of the living body measured by the biological information measurement device, may display the blood pressure value received on the second display unit in a case where it is determined that arrhythmia has been absent during the measurement of the blood pressure of the living body, and may not display the blood pressure value but display the information of the fact that arrhythmia has been present on the second display unit in a case where it is determined that arrhythmia has been present during the measurement of the blood pressure of the living body.

Furthermore, the biological information measurement device may transmit pulse wave information related to the measurement of the blood pressure to the information processing terminal, and

the information processing terminal may include a second display unit, may measure blood pressure of the living body on the basis of the pulse wave information received from the biological information measurement device, may display a blood pressure value measured on the second display unit in a case where it is determined that arrhythmia has been absent during the measurement of the blood pressure of the living body, and may not display the blood pressure value but display the information of the fact that arrhythmia has been present on the second display unit in a case where it is determined that arrhythmia has been present during the measurement of the blood pressure of the living body.

With such a configuration, the user can refer to the blood pressure measurement result with an information processing terminal at hand such as a smartphone, and can recognize the presence of arrhythmia without looking at an inaccurate measurement value in a case where arrhythmia has been detected during the blood pressure measurement.

Furthermore, the present invention can also be regarded as a program for causing the information processing device to function as the above-described information processing terminals, and a computer-readable recording medium in which such a program is non-transiently recorded.

Note that each of configurations and processing described above can be combined with each other to constitute the present invention as long as no technical contradiction occurs.

According to the present invention, it is possible to prevent an inaccurate blood pressure measurement result from being indicated to a user and to notify the user of the arrhythmia detected during the blood pressure measurement.

### BRIEF DESCRIPTION OF THE DRAWINGS

FIG. 1 is a block diagram schematically illustrating a device configuration of a blood pressure measurement device according to a first embodiment;

FIG. 2 is a block diagram illustrating functional modules implemented by a control unit of the blood pressure measurement device according to the first embodiment;

FIG. 3 is a flowchart illustrating a flow of blood pressure measurement processing by the blood pressure measurement device according to the first embodiment;

FIG. 4A is a first diagram illustrating an example of a measurement result display screen when arrhythmia has been detected during blood pressure measurement;

FIG. 4B is a second diagram illustrating an example of the measurement result display screen when arrhythmia has been detected during blood pressure measurement;

FIG. 5 is a flowchart illustrating a flow of processing when a history viewing mode is executed in the blood pressure measurement device according to the first embodiment;

FIG. 6 is a flowchart illustrating a flow of processing when blood pressure measurement is performed in a continuous measurement mode in the blood pressure measurement device according to the first embodiment;

FIG. 7 is a first diagram illustrating an example of a measurement result display screen when the blood pressure measurement is performed in the continuous measurement mode;

FIG. 8 is a second diagram illustrating an example of the measurement result display screen when the blood pressure measurement is performed in the continuous measurement mode;

FIG. 9 is a flowchart illustrating a flow of processing when a past average viewing mode is executed in the blood pressure measurement device according to the first embodiment;

FIG. 10 is a diagram illustrating an example of the measurement result display screen when the blood pressure measurement is performed in the past average viewing mode;

FIG. 11 is a block diagram schematically illustrating a device configuration of a biological information measurement device according to a second embodiment;

FIG. 12 is a block diagram illustrating functional modules implemented by a control unit of the biological information measurement device according to the second embodiment;

FIG. 13 is a block diagram schematically illustrating a device configuration of a biological information measurement device according to a third embodiment;

FIG. 14 is a block diagram illustrating functional modules implemented by a control unit of the biological information measurement device according to the third embodiment;

FIG. 15 is a block diagram schematically illustrating a biological information processing system according to a fourth embodiment;

FIG. 16A is a block diagram illustrating functional modules implemented by a control unit of a biological information measurement device according to the fourth embodiment;

FIG. 16B is a block diagram illustrating functional modules implemented by a control unit of the information processing terminal according to the fourth embodiment;

FIG. 17 is a first flowchart illustrating a flow of processing of each of the biological information measurement device and the information processing terminal in the biological information processing system according to the fourth embodiment; and

FIG. 18 is a second flowchart illustrating a flow of processing of each of the biological information measurement device and the information processing terminal in the biological information processing system according to the fourth embodiment.

### DESCRIPTION OF THE EMBODIMENTS

### First embodiment

Hereinafter, specific embodiments of the present invention will be described on the basis of the drawings. However, the specific detail of each configuration described in the embodiments described below, the arrangement relationship thereof, and the like are not intended to limit the scope of the present invention only to them unless otherwise specified.

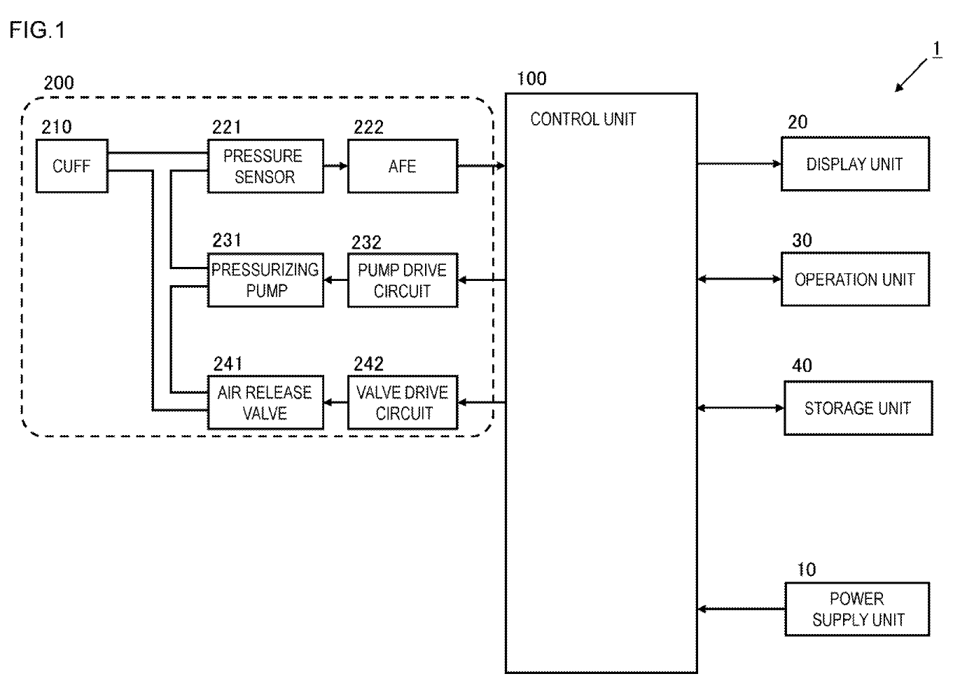

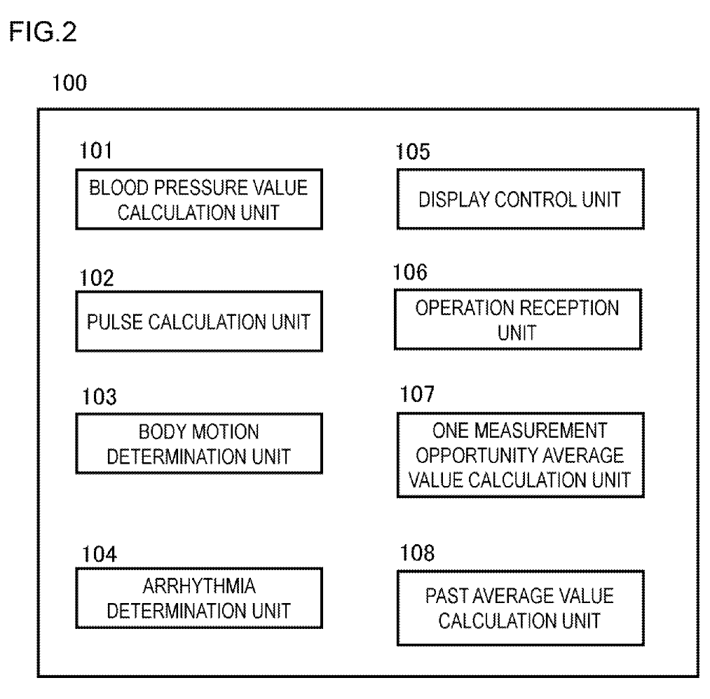

The present invention can be applied to, for example, a blood pressure measurement device 1 as illustrated in FIGS. 1 and 2. FIG. 1 is a block diagram schematically illustrating a device configuration of the blood pressure measurement device 1 in the present embodiment, and FIG. 2 is a block diagram illustrating an example of functional modules implemented by a control unit 100 of the blood pressure measurement device 1.

As illustrated in FIG. 1, the blood pressure measurement device 1 according to the present embodiment schematically includes components such as a power supply unit 10, a display unit 20, an operation unit 30, a storage unit 40, the control unit 100, and a blood pressure measurement unit 200, and has a configuration similar to that of a general portable upper-arm sphygmomanometer including a main body housing, a cuff, and an air tube as an appearance although not illustrated.

The power supply unit 10 is a power source of the blood pressure measurement device 1, and may be, for example, a rechargeable secondary battery such as a lithium ion battery, but is not limited thereto, and may be a primary battery, a terminal connectable to an external power supply, or the like.

The display unit 20 is a functional unit for displaying various types of information, and can be, for example, a display device such as a liquid crystal display (LCD). Furthermore, the operation unit 30 is a functional unit that receives a user operation such as turning on/off of a power supply, execution of measurement, and selection/decision of a menu, and can be configured by an operation switch and the like. Note that a touch panel display can also be adopted as a configuration that serves as both the display unit 20 and the operation unit 30.

The storage unit 40 includes a main storage device such as a random access memory (RAM) and a read only memory (ROM), and an auxiliary storage device such as an HDD and a flash memory, and stores various types of information such as an application program, measurement results such as a blood pressure value and a pulse, information on the presence or absence of arrhythmia to be described later, and other acquired biological information. Note that the measurement results of the blood pressure and the like and other acquired biological information may be stored in the storage unit 40 in association with time information such as the measurement (acquisition) time. As the time information, for example, time information clocked by referring to a real time clock (RTC) of the control unit 100 to be described later can be used. Furthermore, the auxiliary storage device may be configured to be detachable from the main body housing.

The control unit 100 is a functional unit that controls the entire blood pressure measurement device 1, and includes, for example, a processor such as a central processing unit (CPU). When receiving a user's operation via the operation unit 30, the control unit 100 controls each component of the blood pressure measurement device 1 to execute various processing such as blood pressure measurement and presentation of various types of information according to a predetermined program. The predetermined program is stored in the storage unit 40 and read therefrom. As the processor, in addition to the CPU, a graphic processing unit (GPU), a field programmable grid array (FPGA), an application specific integrated circuit (ASIC), or the like can be adopted.

Furthermore, functional modules illustrated in FIG. 2, including a blood pressure value calculation unit 101, a pulse calculation unit 102, a body motion determination unit 103, an arrhythmia determination unit 104, a display control unit 105, an operation reception unit 106, a one measurement opportunity average value calculation unit 107, and a past average value calculation unit 108 are implemented by the control unit 100. These functional modules will be described in detail later.

The blood pressure measurement unit 200 includes a cuff 210, a pressure sensor 221, an analog front end (AFE) 222 including an analog-to-digital (A/D) converter, a pressurizing pump 231, a pump drive circuit 232, an air release valve 241, and a valve drive circuit 242, and is a functional unit that measures the blood pressure of the user by a so-called oscillometric method. The blood pressure measurement by the oscillometric method is a well-known technology, and thus a detailed description thereof will be omitted.

The cuff 210 is a member used with being wound around the upper arm of the user and includes the pressurizing pump 231 (pump drive circuit 232) disposed in the main body housing, an air bladder (not illustrated) communicating with the air release valve 241 (valve drive circuit 242) via an air tube (not illustrated), a belt (not illustrated) incorporating the air bladder, the pressure sensor 221 provided on the belt, and the like.

Note that the belt of the cuff 210 is provided with a fixing unit (for example, a hook-and-loop fastener) for fixing the cuff 210 to the upper arm of the user, and the cuff 210 is wound around the upper arm of the user by the belt when blood pressure measurement is performed using the blood pressure measurement device 1.

For example, a piezoresistive sensor including a piezoelectric element can be adopted as the pressure sensor 221, and at least a pressure pulse wave of the user is acquired. Furthermore, the blood pressure measurement device 1 according to the present embodiment calculates the blood pressure and the pulse of the user on the basis of the pressure pulse wave acquired by the pressure sensor 221, and detects the presence or absence of the body motion and arrhythmia of the user during the blood pressure measurement as described later.

At the time of blood pressure measurement, the control unit 100 controls the pressurizing pump 231 (pump drive circuit 232) and the air release valve 241 (valve drive circuit 242) to adjust the cuff pressure of the cuff 210. Specifically, control is performed such that the pressurizing pump 231 is driven to send air to the cuff 210 to inflate the cuff 210 (increase the cuff pressure). In this way, after the blood flow is once inhibited by compressing the blood vessel of the upper arm of the user, control is performed such that the pressurizing pump 231 is stopped to open the air release valve 241 and the air is gradually released from the cuff 210 to contract the cuff 210 (reduce the cuff pressure).

Next, functional modules illustrated in FIG. 2 will be described. The blood pressure value calculation unit 101 calculates the blood pressure value of the user on the basis of the pressure pulse wave acquired by the pressure sensor 221. Note that, in the present embodiment, the blood pressure value is calculated by the oscillometric method, but it is also possible to provide a microphone in the cuff 210 and calculate the blood pressure value by, for example, a Korotkoff method of detecting a Korotkoff sound.

The pulse calculation unit 102 calculates the pulse rate of the user on the basis of the pressure pulse wave (such as the peak number of the pulse wave) acquired by the pressure sensor 221. The body motion determination unit 103 determines the presence or absence and the number of times of body motion during blood pressure measurement on the basis of the pressure pulse wave (such as disturbance of pulse waveform) acquired by the pressure sensor 221.

Furthermore, the arrhythmia determination unit 104 determines (detects) the presence or absence of arrhythmia during the blood pressure measurement on the basis of the pressure pulse wave (information on the time interval between the peaks of the pulse wave, and the like) acquired by the pressure sensor 221. That is, in the present embodiment, a combination of the arrhythmia determination unit 104 and the pressure sensor 221 corresponds to an arrhythmia information acquisition unit.

The display control unit 105 controls the display unit 20 (the display contents thereof). For example, the display control unit 105 causes the display unit 20 to display menu selection, measurement results, past history data, and the like, and specific display contents will be described later.

The operation reception unit 106 receives an operation input by a user via the operation unit 30. For example, when the user presses a measurement start button, an item selection button, a decision button, or the like (each including an interface expressed on an image), the blood pressure measurement device 1 is controlled to perform the associated operation.

The one measurement opportunity average value calculation unit 107 calculates, in a case where blood pressure measurement is performed a plurality of times (for example, three times) at predetermined intervals (for example, two minutes), an average value of the measured blood pressure values. In the present embodiment, a “continuous measurement mode” selectable by a user operation is implemented. In a case where blood pressure measurement is performed in this mode, three times of continuous measurement are automatically performed at intervals of two minutes, and an average value of three times of measurement is a measurement result of one measurement opportunity. However, even if the normal blood pressure measurement is performed a plurality of times, in a case where the condition of the one measurement opportunity is satisfied, the one measurement opportunity average value calculation unit 107 can calculate an average value afterwards using the data of the plurality of measurement results and use the average value as the measurement result of the one measurement opportunity.

The past average value calculation unit 108 calculates an average value for each time zone (morning/night) in past any period stored in the storage unit 40. For example, the user may select the period from preset periods such as three days, one week, ten days, and one month, or may decide the period on a daily basis. Alternatively, only an average value of a preset predetermined period (for example, last one week) may be calculated.

### Flow of blood pressure measurement processing

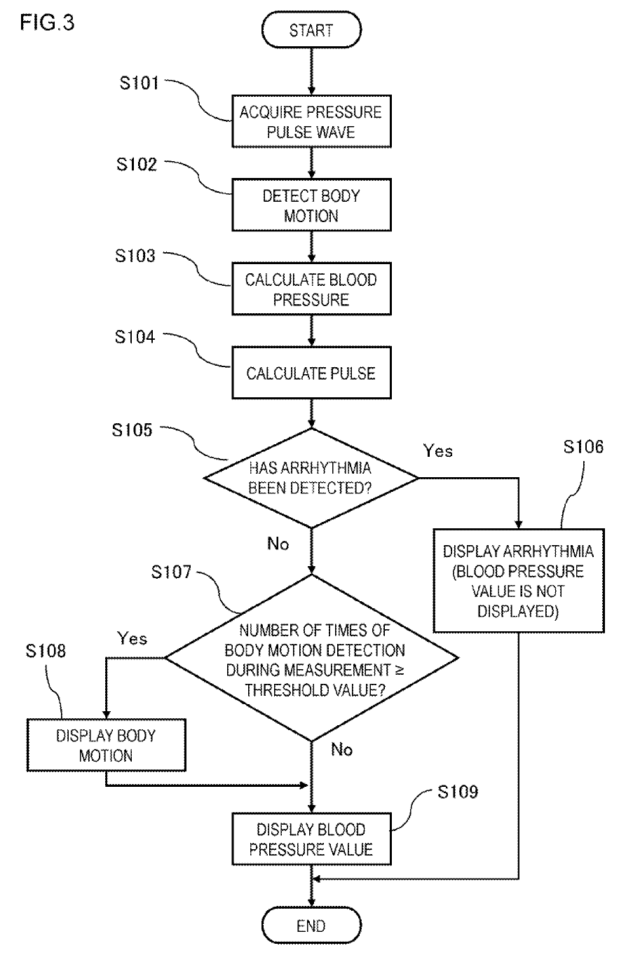

Next, a flow of processing when measurement of blood pressure is executed by the blood pressure measurement device 1 will be described on the basis of FIG. 3. FIG. 3 is a flowchart illustrating a flow of processing by the blood pressure measurement device 1 when measurement of the blood pressure is performed in a normal mode. Prior to the execution of the blood pressure measurement, the user winds the cuff 210 around his/her upper arm, and then instructs execution of the blood pressure measurement in the normal mode via the operation unit 30.

After receiving the instruction of the blood pressure measurement, the blood pressure measurement device 1 acquires the pressure pulse wave of the user by the blood pressure measurement unit 200 (S101). Based on the acquired pressure pulse wave, the control unit 100 (the blood pressure value calculation unit 101, the pulse calculation unit 102, and the body motion determination unit 103) detects the body motion of the user (S102), calculates the blood pressure value (S103), and calculates the pulse rate (S104). In addition, the control unit 100 (arrhythmia determination unit 104) determines the presence or absence of arrhythmia during blood pressure measurement on the basis of the acquired pressure pulse wave (S105).

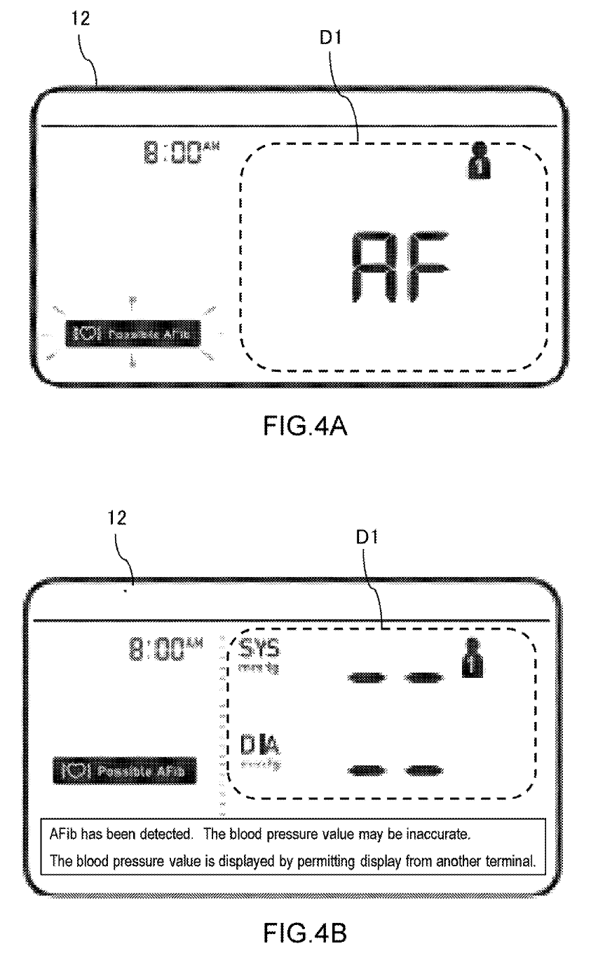

In a case where it is determined in step S105 that arrhythmia has been present during the blood pressure measurement, the control unit 100 (display control unit 105) does not display the calculated blood pressure value but displays, as a measurement result, the fact that arrhythmia has been present on the display unit 20 (S106). FIG. 4A illustrates an example of the display unit 20 when displaying the measurement result of a case where arrhythmia has been detected during the blood pressure measurement in this manner. In the example illustrated in FIG. 4A, in a blood pressure display area D1 for displaying the measured blood pressure value of the display unit 20, the blood pressure value is not displayed, but “AF” indicating that arrhythmia (in this case, atrial fibrillation) has been detected is displayed. Furthermore, a mark indicating that atrial fibrillation has been detected is also displayed in a blinking manner on the lower left side of the screen.

On the other hand, in a case where it is determined in step S105 that arrhythmia has been absent during the blood pressure measurement, the control unit 100 (body motion determination unit 103) determines whether or not the number of times of body motion detected during the blood pressure measurement exceeds a predetermined threshold value (S107). In a case where it is determined in step S107 that the number of times of body motion is equal to or less than the threshold value, the control unit 100 (display control unit 105) displays the blood pressure value in the blood pressure display area D1 of the display unit 20 (S109).

In a case where it is determined in step S107 that the number of times of body motion exceeds the threshold value, the control unit 100 (display control unit 105) displays the fact that body motion has been detected (which may result in the low accuracy of the blood pressure value) in an area other than the blood pressure display area D1 of the display unit 20 (S108), and displays the blood pressure value in the blood pressure display area D1 (S109). Note that step S108 and step S109 may be executed simultaneously.

After displaying the blood pressure measurement result on the display unit 20 in step S106 or step S109, the blood pressure measurement device 1 stores the measurement result in the storage unit 40 in association with the information on the measurement time, and ends the series of processing. As the information to be stored in the storage unit 40 in a case where arrhythmia has been detected during the measurement, the calculated blood pressure value may be stored in association with the information on the arrhythmia, or the calculated blood pressure value may be discarded and only the information on the arrhythmia may be stored.

With the display of the measurement result as described above, in a case where arrhythmia has been detected during the blood pressure measurement, it is possible to prevent an inaccurate blood pressure measurement result from being indicated to the user and to notify the user of the arrhythmia detected during the blood pressure measurement. Furthermore, even in a case where arrhythmia has not been detected, when the number of times of body motion exceeds the threshold value, it is possible to alert the user by displaying that the accuracy of the calculated blood pressure value has decreased together with the blood pressure value.

### Flow of past data display processing

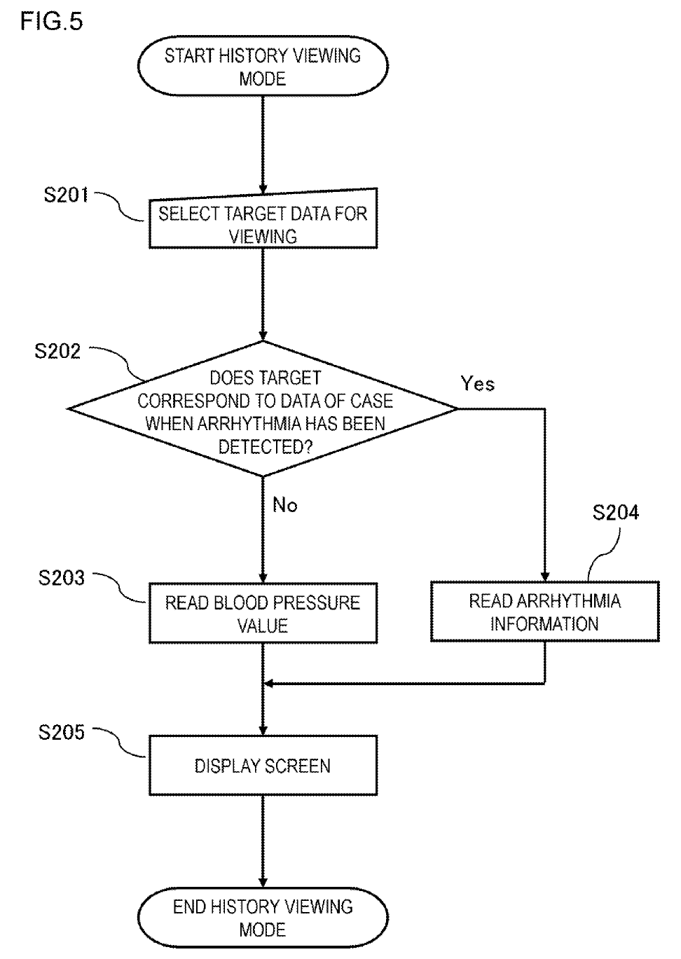

Subsequently, a flow of processing when the past measurement data stored in the storage unit 40 is displayed on the display unit 20 will be described on the basis of FIG. 5. FIG. 5 is a flowchart illustrating a flow of processing of referring to past measurement data (history viewing mode) in the blood pressure measurement device 1. As illustrated in FIG. 5, first, past measurement data (for example, which is read from the storage unit 40 and displayed as a list on the display unit 20) that the user wants to refer to is selected via the operation unit 30 (S201).

Next, the control unit 100 determines whether or not the measurement data according to the user's selection is data of the case where arrhythmia has been detected during the measurement (S202). Specifically, since the measurement data of the case where arrhythmia has been detected is stored in association with the fact that arrhythmia has been detected and the detail of the arrhythmia, it is determined whether or not the selected measurement data corresponds to such data. In a case where it is determined in step S202 that the measurement data according to the selection is not data of the case where arrhythmia has been detected during the measurement, the control unit 100 reads information on the measured blood pressure value (S203), displays the information on the display unit 20 (S205), and ends the series of processing.

On the other hand, in a case where it is determined in step S202 that the measurement data according to the selection is measurement data of the case where arrhythmia has been detected during the measurement, the control unit 100 reads the information on the arrhythmia (S204), displays a measurement result screen (screen as illustrated in FIG. 4A) indicating the fact that arrhythmia has been present without displaying the blood pressure value on the display unit 20 (S205), and ends the series of processing.

In step S204, in a case where the information on the calculated blood pressure value is also stored together with the information on the arrhythmia in the storage unit 40, only the information on the arrhythmia is selected and read without reading the information on the blood pressure value. On the other hand, when the calculated blood pressure value is discarded and only the information on the arrhythmia is stored in the storage unit 40 at the time of blood pressure measurement, since there is no target blood pressure value in the first place, only the stored information on the arrhythmia is read.

According to such processing, the user can view the past measurement result, and even in a case where arrhythmia has been detected at the time of the past measurement, it is possible to prevent an inaccurate blood pressure measurement result from being indicated to the user, and to notify the user of the arrhythmia detected during the blood pressure measurement.

### Flow of processing in continuous measurement mode

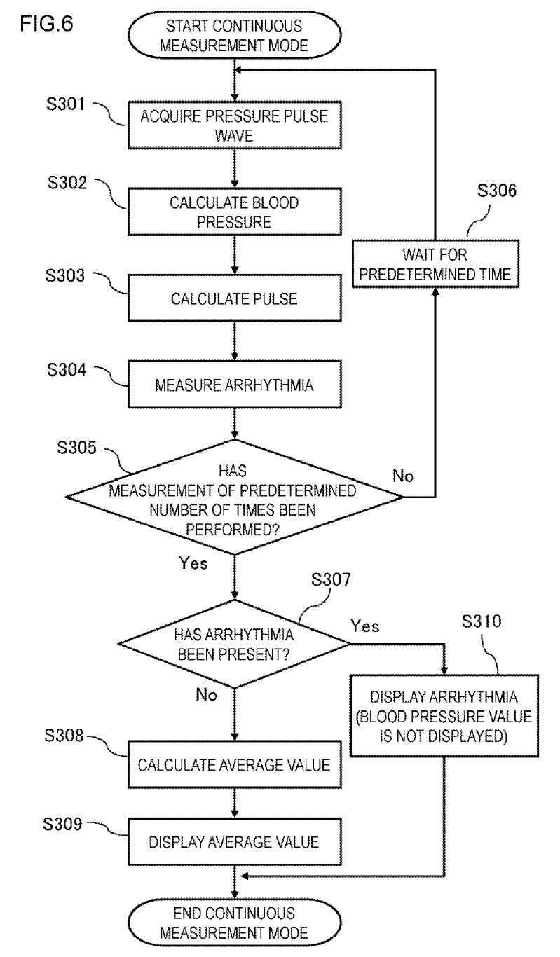

Subsequently, a flow of processing in a case where blood pressure measurement is continuously performed a plurality of times at predetermined intervals will be described on the basis of FIG. 6. FIG. 6 is a flowchart illustrating a flow of processing in a case where the blood pressure measurement device 1 performs blood pressure measurement in the continuous measurement mode. Prior to the execution of the blood pressure measurement, the user winds the cuff 210 around his/her upper arm, and then instructs execution of the blood pressure measurement in the continuous measurement mode via the operation unit 30.

After receiving the instruction of the blood pressure measurement in the continuous measurement mode, the blood pressure measurement device 1 acquires the pressure pulse wave of the user by the blood pressure measurement unit 200 (S301). Furthermore, based on the acquired pressure pulse wave, the control unit 100 (the blood pressure value calculation unit 101, the pulse calculation unit 102, and the body motion determination unit 103) detects the body motion of the user (not illustrated), calculates the blood pressure value (S302), and calculates the pulse rate (S303). In addition, the control unit 100 (arrhythmia determination unit 104) determines the presence or absence of arrhythmia during blood pressure measurement on the basis of the acquired pressure pulse wave (S304). Note that the information on the body motion, the blood pressure value, the pulse rate, and the presence or absence of arrhythmia is recorded in the storage unit 40 every time the measurement is performed.

Next, the control unit 100 determines whether or not the blood pressure measurement as one measurement opportunity has been performed a predetermined number of times (S305). In a case where it is determined in step S305 that the measurement for the predetermined number of times has not been performed yet, the control unit 100 waits for a predetermined time (for example, 2 minutes) and then returns to step S301 to repeat the subsequent processing.

On the other hand, in a case where it is determined in step S305 that the measurement for the predetermined number of times has been performed, the control unit 100 continuously determines whether or not arrhythmia has been detected in any of the plurality of times of measurement (S307). In a case where it is determined in step S307 that arrhythmia has not been detected in any of the plurality of measurements, the control unit 100 (one measurement opportunity average value calculation unit 107) calculates an average value of the blood pressure values calculated in the plurality of times of measurement (S308). Then, the control unit 100 (display control unit 105) displays the calculated average value on the display unit 20 (S309), and ends the series of processing.

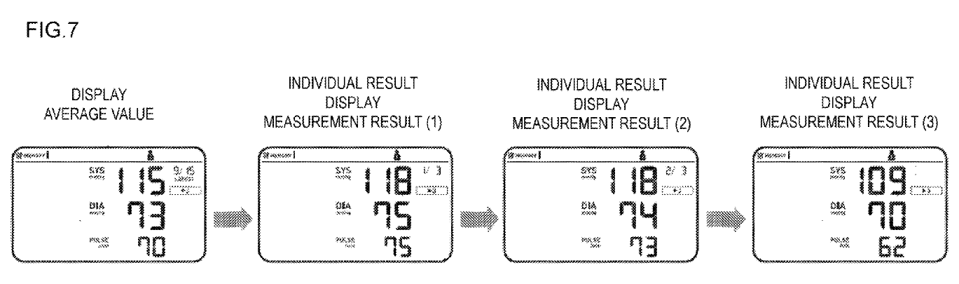

FIG. 7 is an explanatory diagram illustrating a screen display example of the blood pressure measurement result in a case where the measurement has been performed in the continuous measurement mode. The screen of “AVERAGE VALUE DISPLAY” at the left end of FIG. 7 is a screen displaying the measurement result as one measurement opportunity, and is an example of the screen displayed in step S309. In a case where the measurement has been performed in the continuous measurement mode, a user operation allows transition from the average value display to screens respectively showing individual measurement results. As illustrated in FIG. 7, the screen is transitioned from the “AVERAGE VALUE DISPLAY” to the display of the first measurement result, the display of the second measurement result, and the display of the third measurement result in this order to allow viewing of the individual measurement results.

On the other hand, in a case where it is determined in step S307 that arrhythmia has been detected in any of the plurality of measurements, the control unit 100 (display control unit 105) does not display the average value but displays information of the fact that arrhythmia has been present on the display unit 20 (S310), and ends the series of processing.

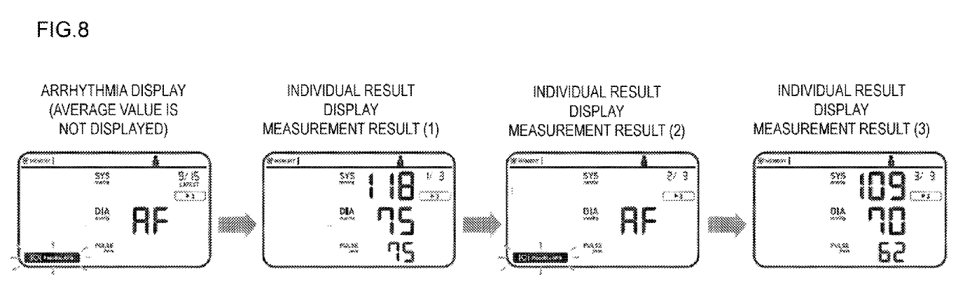

FIG. 8 is an explanatory diagram illustrating a screen display example of the blood pressure measurement result of a case where, when the measurement has been performed in the continuous measurement mode, arrhythmia has been detected at the time of any measurement. The screen of “ARRHYTHMIA DISPLAY” at the left end of FIG. 8 is a screen displaying the measurement result as one measurement opportunity, and is an example of the screen displayed in step S310. Even in a case where arrhythmia has been detected at the time of any measurement, a user operation allows transition to the screens respectively showing individual measurement results. As illustrated in FIG. 8, the screen is transitioned from the “ARRHYTHMIA DISPLAY” to the display of the first measurement result, the display of the second measurement result, and the display of the third measurement result in this order to allow viewing of the individual measurement results.

According to the processing as described above, even in a case where the measurement value of one measurement opportunity is indicated using the average value of measurement values from the plurality of times of measurement, it is possible to prevent an inaccurate blood pressure measurement result from being indicated to the user and to promptly notify the user of arrhythmia.

### Flow of past average value display processing

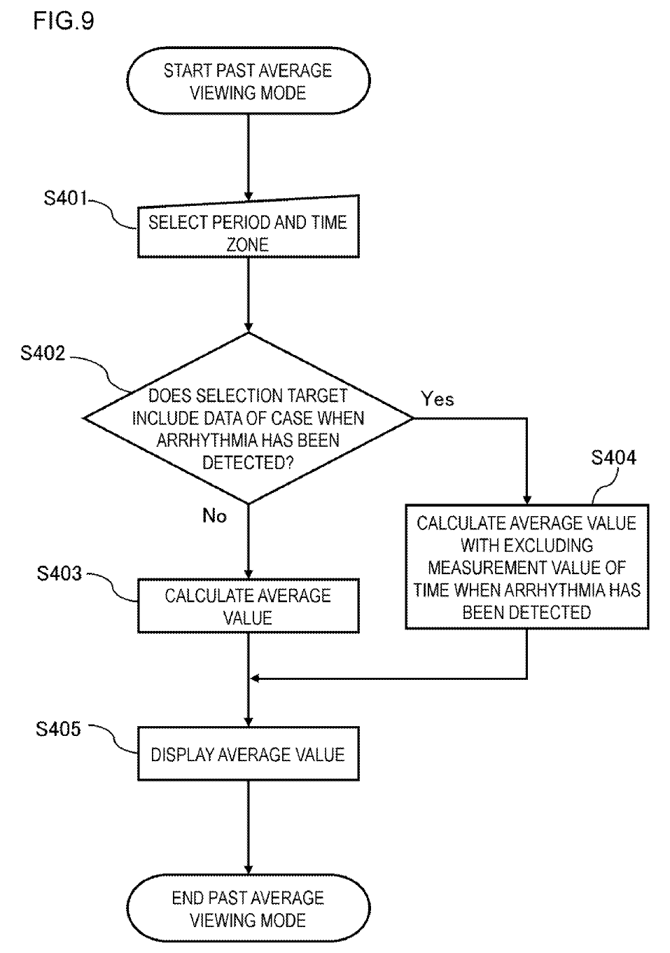

Next, a flow of processing in the blood pressure measurement device 1 in a case where the average value of the blood pressures in past any period is viewed will be described on the basis of FIG. 9. FIG. 9 is a flowchart illustrating a flow of processing of referring to an average value of the blood pressures measured in past any period (past average viewing mode) by the blood pressure measurement device 1. As illustrated in FIG. 9, first, a past period and a time zone (for example, morning/night) that the user wants to refer to are selected via the operation unit 30 (S401).

Thereafter, the control unit 100 determines whether or not measurement data for the target period/time zone selected by the user includes data of the case where arrhythmia has been detected (S402). In a case where it is determined in step S402 that the measurement data in a range selected by the user does not include measurement data of the case where arrhythmia has been detected during the measurement, the control unit 100 (past average value calculation unit 108) calculates an average value of a plurality of pieces of measurement data to be selected (S403). Then, the control unit 100 (display control unit 105) displays the calculated average value on the display unit 20, and ends the series of processing.

On the other hand, in a case where it is determined in step S402 that the measurement data in a range selected by the user includes measurement data of the case where arrhythmia has been detected during the measurement, the control unit 100 (past average value calculation unit 108) excludes the measurement data of the case where arrhythmia has been detected to calculate the average value (S404). Then, the control unit 100 (display control unit 105) displays the calculated average value on the display unit 20, and ends the series of processing.

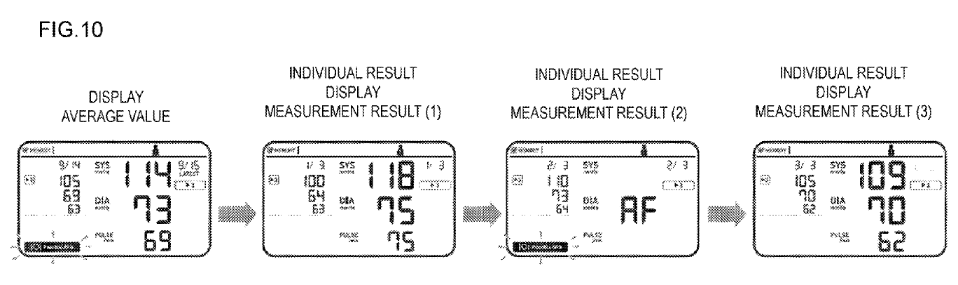

FIG. 10 is an explanatory diagram illustrating a screen display example displayed in a case where, in the past average viewing mode, the measurement data in the range selected by the user includes measurement data of the case where arrhythmia has been detected during the measurement. The screen of “AVERAGE VALUE DISPLAY” at the left end of FIG. 10 is a screen showing the average value calculated by excluding, from the measurement data in the period selected by the user, the measurement data of the case where arrhythmia has been detected. Also in the past average viewing mode, as illustrated in FIG. 10, a user operation allows transition to screens respectively displaying individual measurement results in addition to the display indicating the average value.

According to such processing, the user can view the average value for each time zone in past any period, and it is possible to prevent the average value from being calculated using the inaccurate measurement value of the case where arrhythmia has been detected.

### Modification

In the above embodiment, in a case where arrhythmia has been detected during the blood pressure measurement, the fact that arrhythmia has been detected is displayed without displaying the blood pressure value, thereby preventing the inaccurate blood pressure measurement result from being indicated to the user. However, if the user recognizes that arrhythmia has been detected during the measurement and knows that the measured blood pressure value is inaccurate, the blood pressure value may be displayed as the reference information.

Specifically, for example, the control unit 100 (display control unit 105) performs display as illustrated in FIG. 4B as a measurement result screen of the case where arrhythmia has been detected during the blood pressure measurement. In the screen display example illustrated in FIG. 4B, in the blood pressure display area D1 for displaying the measured blood pressure value, the blood pressure value is not displayed. On the other hand, in an area other than the blood pressure display area D1, the fact that arrhythmia (AFib) has been detected and the fact that the calculated blood pressure value is inaccurate are informed, and operation guidance in the case of viewing the blood pressure value knowing the facts is displayed.

Then, in a case where the decision button is pressed (that is, in a case where the control unit 100 (operation reception unit 106) receives an operation to display the blood pressure value knowing that the value is inaccurate) after the screen as illustrated in FIG. 4B is displayed, control is performed such that the screen transitions to a screen showing the blood pressure value of the case where arrhythmia has been detected during the measurement. As a result, the user can recognize the presence of arrhythmia, and can view the measured blood pressure as the reference value knowing the inaccuracy.

### Second embodiment

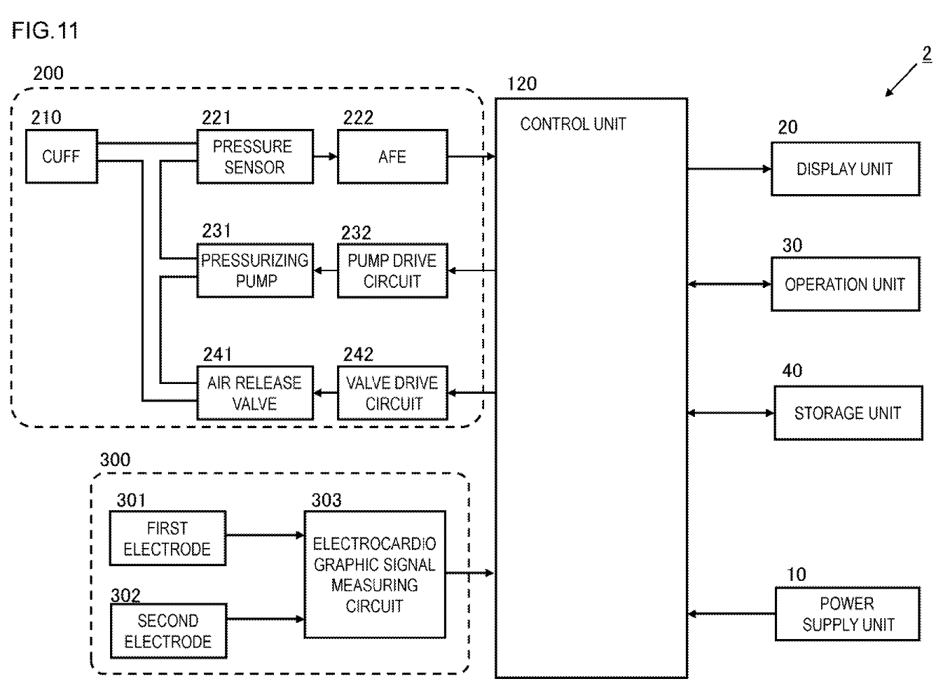

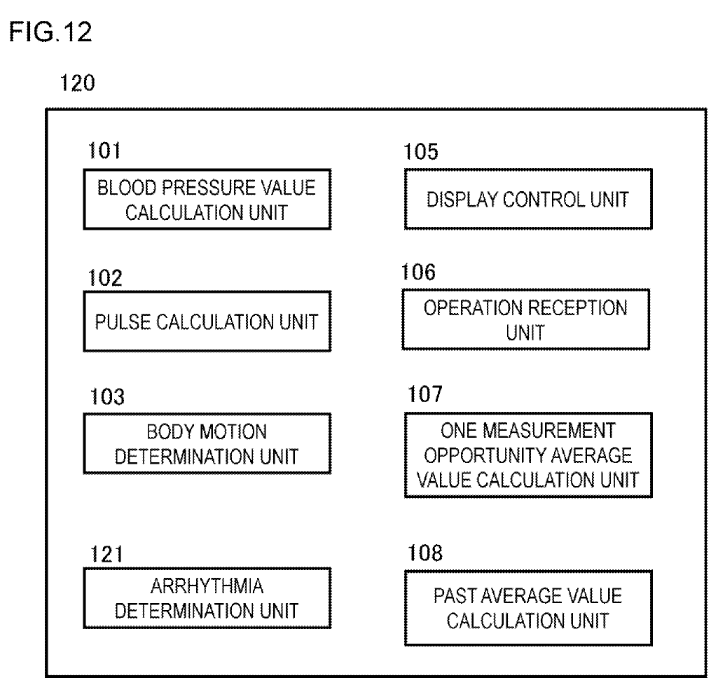

Next, still another embodiment of the present invention will be described on the basis of FIGS. 11 and 12. A biological information measurement device 2 according to the present embodiment is a hybrid biological information measurement device capable of measuring not only blood pressure but also an electrocardiographic waveform. Note that, since the biological information measurement device 2 includes many configurations common to those of the blood pressure measurement device 1 of the first embodiment, hereinafter, the same reference numerals and signs are used for the same configurations as those of the first embodiment, and repeated description will be omitted.

As illustrated in FIG. 11, the biological information measurement device 2 includes an electrocardiographic waveform measurement unit 300 in addition to the same configurations as those of the blood pressure measurement device 1. The electrocardiographic waveform measurement unit 300 includes a plurality of electrodes (a first electrode 301 and a second electrode 302) and an electrocardiographic signal measuring circuit 303. The electrocardiographic signal measuring circuit 303 is a circuit for measuring an electrocardiographic waveform on the basis of an electrocardiographic signal obtained on the basis of a potential difference between two electrodes in contact with a living body, and includes an amplifier, a filter, an A/D conversion circuit, and the like.

Although not illustrated, the first electrode 301 and the second electrode 302 are disposed at positions separated from each other in the main body housing. When the user performs measurement processing in a state where one of the left and right fingers is in contact with the first electrode 301 and the other is in contact with the second electrode 302, the potential difference between the first electrode 301 and the second electrode 302 is measured as an electrocardiographic waveform by the electrocardiographic signal measuring circuit 303 (so-called I induction), and the electrocardiographic waveform (electrocardiogram) is measured by recording the potential difference.

Furthermore, as illustrated in FIG. 12, the biological information measurement device 2 includes functional modules including the blood pressure value calculation unit 101, the pulse calculation unit 102, the body motion determination unit 103, the display control unit 105, the one measurement opportunity average value calculation unit 107, the past average value calculation unit 108, and an arrhythmia determination unit 121 which are implemented by a control unit 120.

In the present embodiment, the arrhythmia determination unit 121 detects arrhythmia on the basis of not the pressure pulse wave but the electrocardiographic waveform measured by the electrocardiographic signal measuring circuit 303. Specifically, by simultaneously performing the blood pressure measurement by the blood pressure measurement unit 200 and the electrocardiographic waveform measurement by the electrocardiographic waveform measurement unit 300, the arrhythmia determination unit 121 determines the presence or absence of arrhythmia during the blood pressure measurement on the basis of the electrocardiographic waveform acquired during the blood pressure measurement. That is, in the present embodiment, a combination of the arrhythmia determination unit 121 and the electrocardiographic waveform measurement unit 300 corresponds to the arrhythmia information acquisition unit. Furthermore, the display control unit 105 causes the display unit 20 to display a graph display of the acquired electrocardiographic waveform in response to a user operation.

Note that the flow of processing of the blood pressure measurement in the biological information measurement device 2 according to the present embodiment is similar to that of the blood pressure measurement device 1 of the first embodiment except that the detection of arrhythmia is performed on the basis of the electrocardiographic waveform instead of the pressure pulse wave, and the display contents of the blood pressure measurement result, the viewing of the past history data of the blood pressure values, and the like are also similar to those of the blood pressure measurement device 1.

As in the biological information measurement device 2 according to the present embodiment, by detecting arrhythmia on the basis of the electrocardiographic waveform, it is possible to more accurately detect arrhythmia during the blood pressure measurement.

### Third embodiment

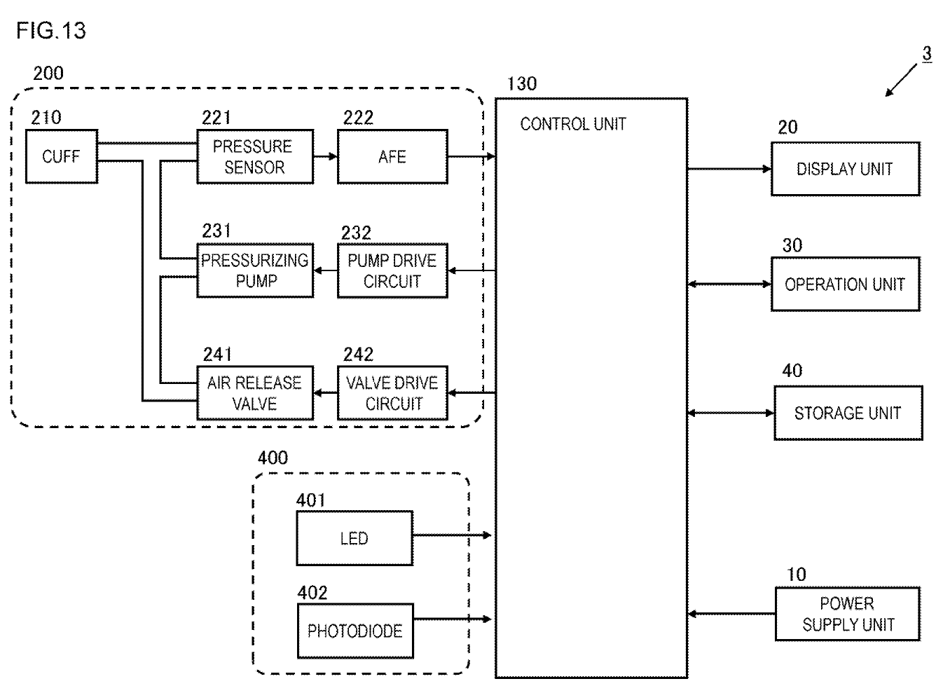

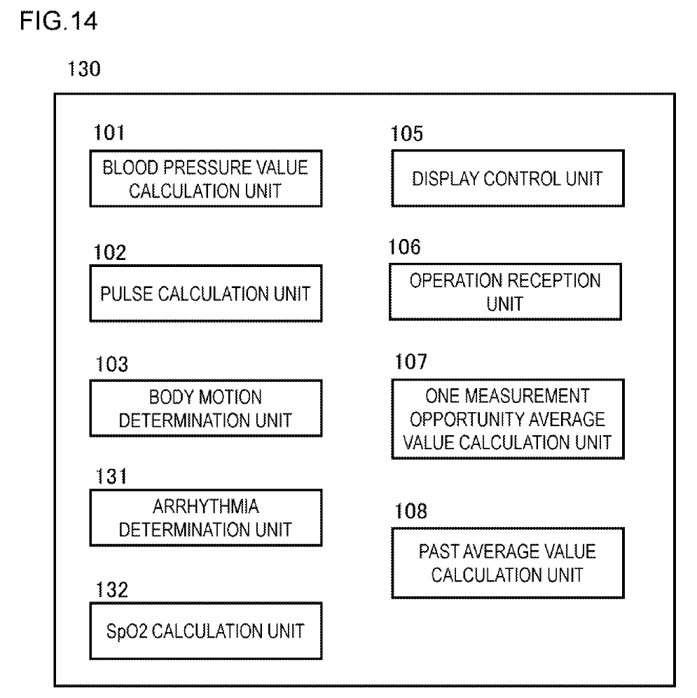

Next, still another embodiment of the present invention will be described on the basis of FIGS. 13 and 14. A biological information measurement device 3 according to the present embodiment is a hybrid biological information measurement device capable of measuring not only blood pressure but also blood oxygen saturation (SpO2).

As illustrated in FIG. 13, the biological information measurement device 3 includes an SpO2 measurement unit 400 in addition to the same configurations as those of the blood pressure measurement device 1. The SpO2 measurement unit 400 includes an LED 401 as a light emitting element and a photodiode 402 as a light receiving element, and acquires information for calculating blood oxygen saturation by receiving reflected light of red light or infrared light emitted from the LED 401 by the photodiode 402.

Note that the SpO2 measurement unit 400 can not only measure the blood oxygen saturation but also detect a change in the blood flow rate accompanying the heartbeat as a change in the reflected light received by the photodiode 402 and acquire the pulse wave on the basis of the change.

Furthermore, as illustrated in FIG. 14, the biological information measurement device 3 includes functional modules including the blood pressure value calculation unit 101, the pulse calculation unit 102, the body motion determination unit 103, the display control unit 105, the one measurement opportunity average value calculation unit 107, the past average value calculation unit 108, the arrhythmia determination unit 131, and an SpO2 calculation unit 132 which are implemented by a control unit 130.

The SpO2 calculation unit 132 calculates the blood oxygen concentration using the information acquired by the SpO2 measurement unit 400 (the intensity of the reflected light received by the photodiode 402).

Furthermore, in the present embodiment, the arrhythmia determination unit 131 detects arrhythmia on the basis of not the pressure pulse wave but the photoelectric pulse wave acquired by the SpO2 measurement unit 400. Specifically, by simultaneously performing the blood pressure measurement by the blood pressure measurement unit 200 and the measurement by the SpO2 measurement unit 400, the arrhythmia determination unit 131 determines the presence or absence of arrhythmia during the blood pressure measurement on the basis of the pulse wave information acquired by the SpO2 measurement unit 400. That is, in the present embodiment, a combination of the arrhythmia determination unit 131 and the SpO2 measurement unit 400 corresponds to the arrhythmia information acquisition unit.

Note that the flow of processing of the blood pressure measurement in the biological information measurement device 3 according to the present embodiment is similar to that of the blood pressure measurement device 1 of the first embodiment except that the detection of arrhythmia is performed on the basis of the photoelectric pulse wave instead of the pressure pulse wave, and the display contents of the blood pressure measurement result, the viewing of the past history data of the blood pressure values, and the like are also similar to those of the blood pressure measurement device 1.

### Fourth embodiment

### System configuration

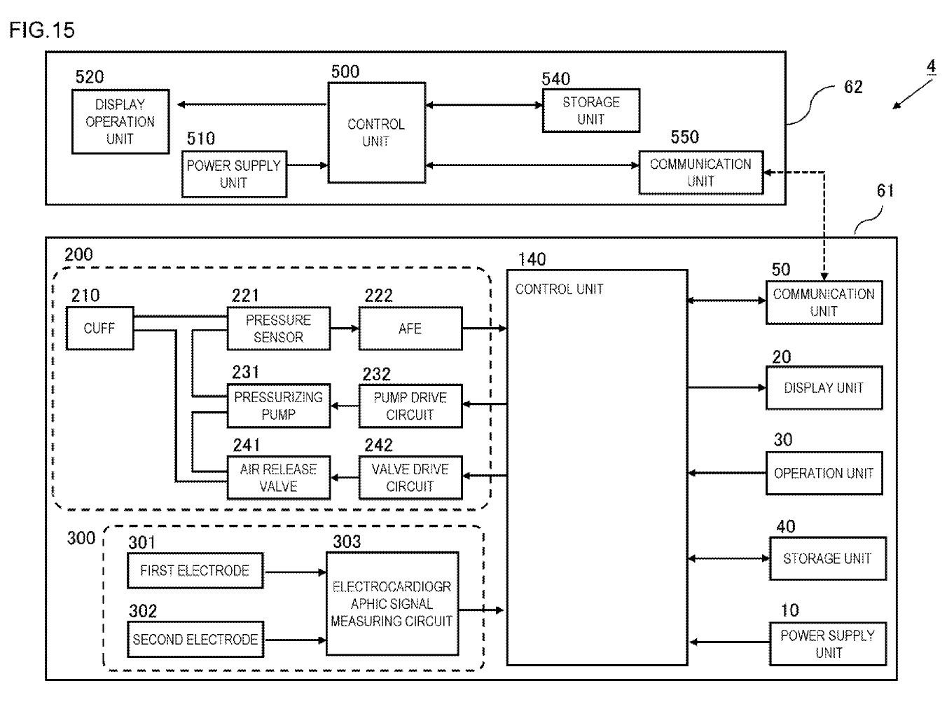

In each of the above embodiments, the detection of arrhythmia during the blood pressure measurement (determination of the presence or absence of arrhythmia) is executed in the biological information measurement device, but the detection of arrhythmia during the blood pressure measurement may be executed in another device. That is, the present invention is also applicable as a system including a biological information measurement device and an information processing terminal as in an embodiment described below. FIG. 15 is a block diagram schematically illustrating a configuration of a biological information processing system 4 according to the present embodiment.

As illustrated in FIG. 15, the biological information processing system 4 according to the present embodiment includes a biological information measurement device 61 including the blood pressure measurement unit 200 and the electrocardiographic waveform measurement unit 300, and a smartphone 62 as an example of the information processing terminal, which are configured to be communicably connectable.

The biological information measurement device 61 has a hardware configuration substantially similar to that of the biological information measurement device 2 according to the second embodiment, and is only different in including a communication unit 50. The communication unit 50 functions to communicate with and connect to the smartphone 62, and can adopt an appropriate communication interface according to a communication network to be connected, such as a wired communication port or a wireless communication antenna. Note that a communication method between the biological information measurement device 61 and the smartphone 62 is not particularly limited, and for example, pairing by Bluetooth (registered trademark) low energy (BLE) communication, communication by Wi-Fi (registered trademark) (LAN), or the like can be adopted.

As illustrated in FIG. 15, the smartphone 62 includes configurations such as a power supply unit 510, a display operation unit 520, a storage unit 540, a communication unit 550, and a control unit 500.

The power supply unit 510 is a power source of the smartphone 62, and can be, for example, a rechargeable secondary battery such as a lithium ion battery. The display operation unit 520 can be a touch panel display or the like having both functions of information display and input operation reception.

The storage unit 540 includes a main storage device such as a RAM and a ROM, and an auxiliary storage device such as a flash memory, and stores various types of information such as an application program and various pieces of biological information. Note that the measurement results of the blood pressure and the like and other acquired biological information may be stored in the storage unit 540 in association with time information such as the measurement (acquisition) time. As the time information, for example, time information clocked by referring to an RTC of the control unit 500 can be used. Furthermore, the auxiliary storage device may be configured to be detachable from the main body housing (not illustrated).

The communication unit 550 functions to communicate with and connect to the biological information measurement device 61, and can adopt an appropriate communication interface according to a communication network to be connected, such as a wired communication port or a wireless communication antenna.

The control unit 500 is a functional unit that controls the smartphone, and includes, for example, a processor such as a CPU and a GPU. When receiving a user's operation via the display operation unit 520, the control unit 500 controls each component of the smartphone to exhibit various functions according to a predetermined application program. Note that the predetermined program is stored in the storage unit 540 and read therefrom.

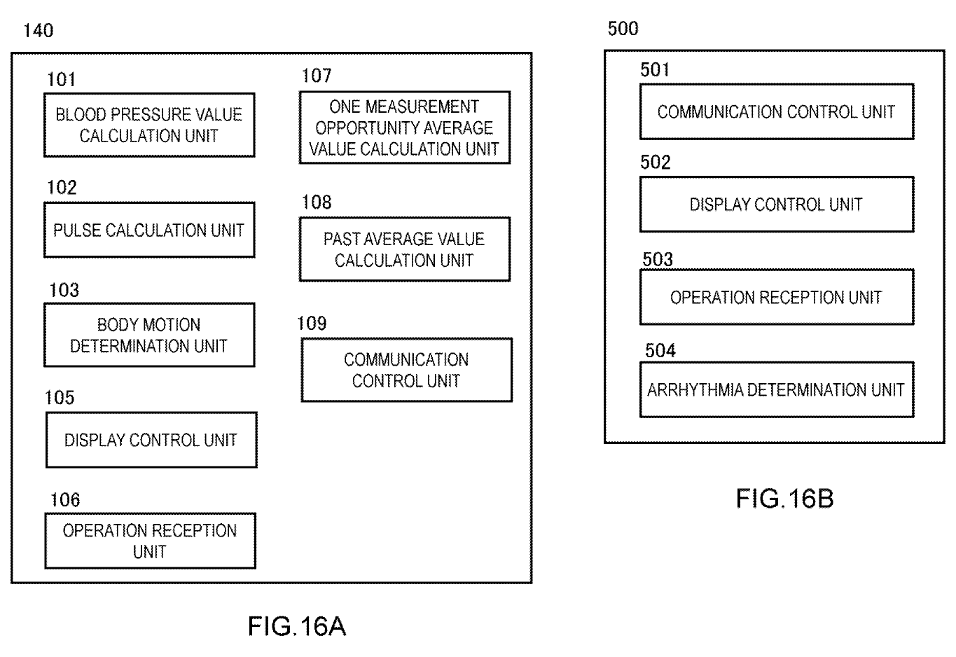

FIG. 16A is a block diagram illustrating part of functional modules implemented by a control unit 140 of the biological information measurement device 61, and FIG. 16B is a block diagram illustrating part of functional modules implemented by the control unit 500 of the smartphone 62.

As illustrated in FIG. 16A, the functional modules of the biological information measurement device 61 are different from those of the biological information measurement device 2 of the second embodiment in that a communication control unit 109 is added and the arrhythmia determination unit does not exist. The communication control unit 109 executes communication with an external device (for example, the smartphone 62) by the communication unit 50.

As illustrated in FIG. 16B, the control unit 500 of the smartphone 62 implements functional modules including a communication control unit 501, a display control unit 502, and an arrhythmia determination unit 504. The communication control unit 501 executes communication with an external device (for example, the biological information measurement device 61) by the communication unit 550.

The display control unit 502 controls the display contents of the display operation unit 520. For example, the display control unit 502 causes the display operation unit 520 to display a menu screen (operation reception screen), guidance related to the progress of biological information measurement, a measurement result of biological information, and the like according to an application program related to cooperation with the biological information measurement device 61.

The operation reception unit 503 receives an input of a user operation via a user interface displayed on the display operation unit 520. For example, in accordance with an application program related to cooperation with the biological information measurement device 61, the operation reception unit 503 receives an operation input such as start of measurement, selection of an item, and screen transition by the biological information measurement device 61.

As will be described later, the arrhythmia determination unit 504 determines (detects), on the basis of the information on the electrocardiographic waveform transmitted from the biological information measurement device 61, the presence or absence of arrhythmia at the time of blood pressure measurement performed simultaneously with the acquisition of the electrocardiographic waveform. That is, in the present embodiment, the detection processing of arrhythmia during the blood pressure measurement is performed not by the biological information measurement device 61 but by the smartphone 62. Then, the information on the presence or absence of arrhythmia determined by the arrhythmia determination unit 504 is transmitted from the communication unit 550 and received by the communication unit 50 of the biological information measurement device 61. That is, in the biological information measurement device 61 in the present embodiment, the communication unit 50 corresponds to the arrhythmia information acquisition unit.

### Flow of processing

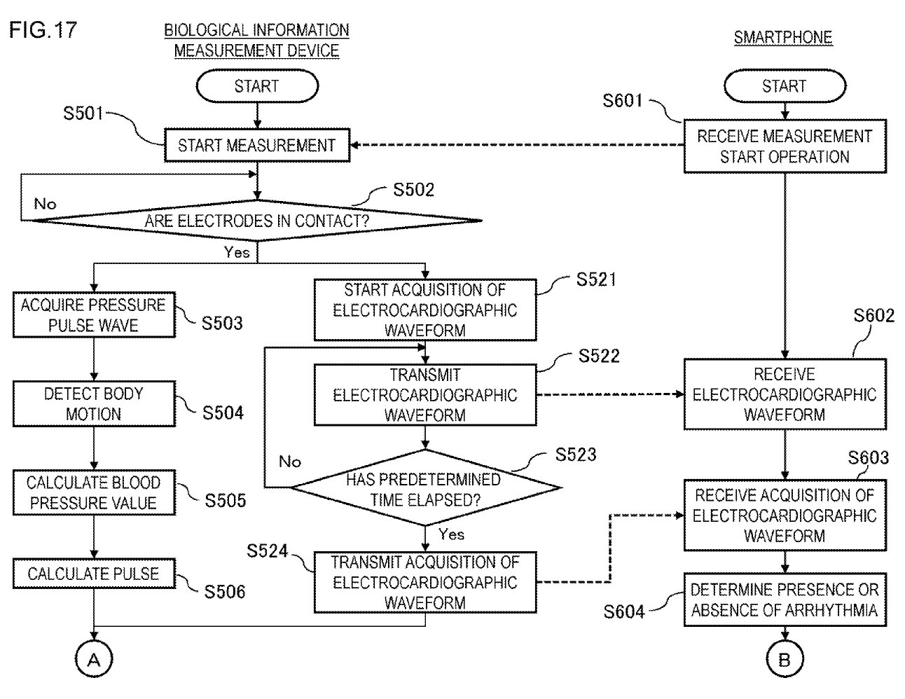

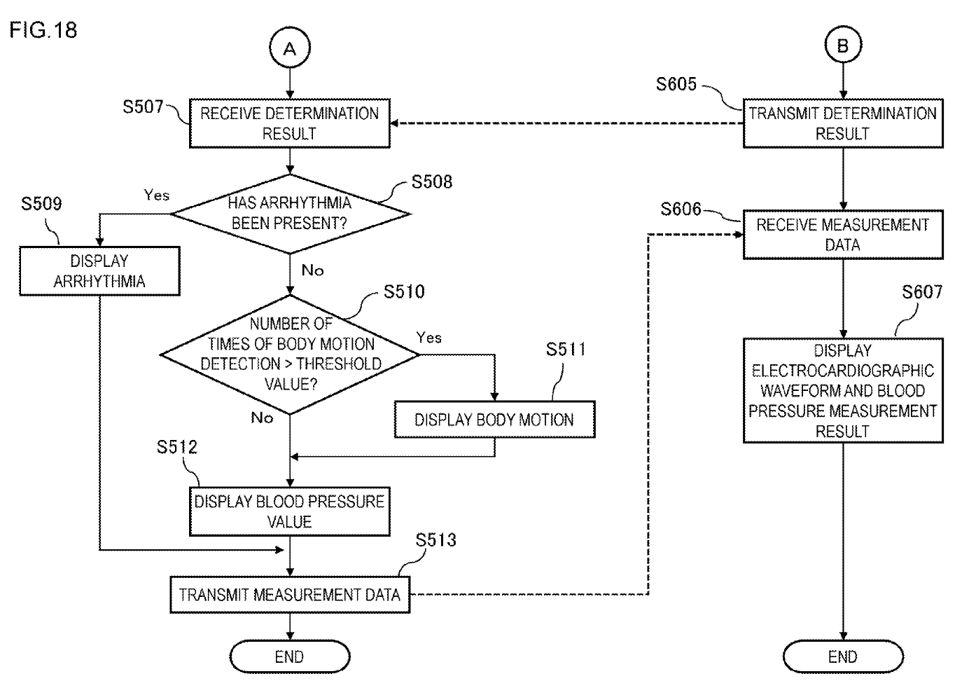

Subsequently, based on FIGS. 17 and 18, flows of blood pressure/electrocardiogram collective measurement processing executed in the biological information processing system 4 according to the present embodiment and information will be described. In the biological information processing system 4 according to the present embodiment, the smartphone 62 can instruct to start the collective measurement of the blood pressure and the electrocardiographic waveform. Therefore, the control unit 500 (operation reception unit 503) of the smartphone 62 receives the operation input of starting the collective measurement of the blood pressure and the electrocardiogram via the display operation unit 520 (S601). The instruction by the operation input is transmitted from the smartphone 62 to the biological information measurement device 61 via the communication unit 550, and the biological information measurement device 61 starts the collective measurement of the blood pressure and the electrocardiogram (S501).

When the collective measurement is started, the biological information measurement device 61 determines whether or not the first electrode 201 and the second electrode 202 are correctly in contact with the living body on the basis of the signal acquired by the electrocardiographic waveform measurement unit 300 (S502). Here, in a case where it is determined that both electrodes are not correctly in contact with the living body, the processing to determine the electrode contact is repeatedly performed. On the other hand, in a case where it is determined in step S502 that both electrodes are correctly in contact with the living body, the biological information measurement device 61 executes acquisition of the pressure pulse wave by the blood pressure measurement unit 200 and acquisition of the electrocardiographic waveform by the electrocardiographic waveform measurement unit 300 in parallel (S503, S521).

The electrocardiographic waveform acquired in step S521 by the biological information measurement device 61 is transmitted to the smartphone 62 via the communication unit 50 (S522), and the smartphone 62 receives the electrocardiographic waveform (S602). The biological information measurement device 61 (control unit 140) continues to acquire the electrocardiographic waveform, and determines whether or not a predetermined time (for example, time sufficient for pulse wave acquisition for blood pressure measurement) has elapsed (S523). Here, in a case where it is determined that the predetermined time has not elapsed, the process returns to step S522, and the subsequent processing is repeated.

On the other hand, in a case where it is determined in step S523 that the predetermined time has elapsed, the biological information measurement device 61 ends the acquisition of the electrocardiographic waveform and transmits that fact to the smartphone 62 via the communication unit 50 (S524), and the smartphone 62 receives the information of the fact that the acquisition of the electrocardiographic waveform has ended (S603). Then, in the smartphone 62 that has received the electrocardiographic waveform for the predetermined time, the control unit 500 (arrhythmia determination unit 504) executes analysis processing of determining (detecting) the presence or absence of arrhythmia during the blood pressure measurement (during pressure pulse wave acquisition) on the basis of the electrocardiographic waveform (S604).

On the other hand, in the biological information measurement device 61, in parallel with the acquisition of the electrocardiographic waveform, acquisition of a pressure pulse wave for blood pressure measurement is performed by the blood pressure measurement unit 200 (S503). In addition, based on the acquired pressure pulse wave, the control unit 140 (the blood pressure value calculation unit 101, the pulse calculation unit 102, and the body motion determination unit 103) detects the body motion of the user (S504), calculates the blood pressure value (S505), and calculates the pulse rate (S506).

Next, referring to FIG. 18, the smartphone 62 transmits the determination result of the presence or absence of arrhythmia performed in step S604 to the biological information measurement device 61 via the communication unit 550 (S605), and the biological information measurement device 61 receives the determination result (S507). Then, in a case where the determination result received in step S507 indicates the presence of arrhythmia during the blood pressure measurement, the biological information measurement device 61 (display control unit 105) does not display the calculated blood pressure value but displays, as a measurement result, the fact that arrhythmia has been present on the display unit 20 (S508, S509).

On the other hand, in a case where the received determination result is the absence of arrhythmia during the blood pressure measurement, the biological information measurement device 61 (body motion determination unit 103) subsequently determines whether or not the number of times of body motion detected during the blood pressure measurement exceeds a predetermined threshold value (S508, S510). Then, in a case where it is determined in step S510 that the number of times of body motion is equal to or less than the threshold value, the biological information measurement device 61 (display control unit 105) displays the blood pressure value and the pulse rate on the display unit 20 (S512).

In a case where it is determined in step S510 that the number of times of body motion exceeds the threshold value, the biological information measurement device 61 (display control unit 105) displays the fact that body motion has been detected in an area other than the blood pressure display area D1 of the display unit 20 (S511), and displays the blood pressure value in the blood pressure display area D1 (S512). Note that step S511 and step S512 may be executed simultaneously.

Thereafter, the biological information measurement device 61 transmits the calculated blood pressure value, pulse rate, and number of times of body motion detection (hereinafter, collectively referred to as blood pressure measurement data) to the smartphone 62 via the communication unit 50 (S513), and ends the series of processing. On the other hand, the smartphone 62 receives the blood pressure measurement data (S606), displays the blood pressure measurement data and the electrocardiographic waveform (electrocardiogram) on the display operation unit 520 (S607), and ends the series of processing.

Note that the order of the processing described above can be appropriately changed or omitted, and for example, the transmission of the blood pressure measurement data from the biological information measurement device 61 is not limited to after step S512, and may be executed at other timings such as after step S507. Furthermore, the smartphone 62 may end the series of processing after step S605 without transmitting the blood pressure measurement data from the biological information measurement device 61.

Furthermore, since it is undetermined which of the processing of the blood pressure value calculation and the processing of the arrhythmia presence or absence determination ends first, step S507 (and step S605) may be executed before the blood pressure value calculation in step S505.

In the above example, in step S523, the biological information measurement device 61 determines whether or not the predetermined time has elapsed from the start of acquisition of the electrocardiographic waveform. However, this processing may be executed on the smartphone 62 side. In this case, the smartphone 62 may transmit a command to end the acquisition of the electrocardiographic waveform to the biological information measurement device 61 after the predetermined time elapses.

In the smartphone 62, after the electrocardiographic waveform is received in step S602, the electrocardiographic waveform may be displayed on the display operation unit 602 in real time.

According to the biological information processing system 4 as described above, the determination of the presence or absence of arrhythmia during the blood pressure measurement can be executed by the information processing terminal such as the smartphone 62 instead of the biological information measurement device 61. As a result, the calculation load of the biological information measurement device can be reduced, and even if a high-performance calculation device is not used, it is possible to prevent an inaccurate blood pressure measurement result from being indicated to the user and to notify the user of the arrhythmia detected during the blood pressure measurement.

### Modification

In the biological information processing system 4, in a case where arrhythmia has been detected during the blood pressure measurement, the fact that arrhythmia has been detected is displayed on the display unit 20 or the display operation unit 520 without displaying the blood pressure value. However, similarly to the modification of the first embodiment, it is possible to adopt the configuration in which the measured blood pressure value is indicated as the reference value.

For example, after the display unit 20 of the biological information measurement device 61 displays in step S509 the fact that arrhythmia is present, the display control unit 502 of the smartphone 62 causes the display operation unit 520 to display the fact that arrhythmia has been detected during the blood pressure measurement, the fact that the measured blood pressure value is inaccurate, and an interface (button icon) indicating whether or not to display the measured blood pressure value as the reference value knowing the facts. Then, when the operation reception unit 503 receives a user operation to display the blood pressure value, the operation reception unit 503 may transmit a signal to display the blood pressure value to the biological information measurement device 61 so that the biological information measurement device 61 displays the blood pressure value on the display unit 20.

Similarly, in the smartphone 62, in a case where arrhythmia has been present during the measurement, the blood pressure value is not displayed in step S607, the fact that arrhythmia has been detected during the blood pressure measurement, the fact that the measured blood pressure value is inaccurate, and an interface indicating whether or not to display the measured blood pressure value as the reference value knowing the facts are displayed, and the blood pressure value may be displayed after the display permission operation is performed.

### Others

Note that the description of the above-described embodiments is merely illustrative of the present invention, and the present invention is not limited to the above-described specific forms. The present invention can be variously modified and combined within the scope of the technical idea. For example, the configuration according to the second embodiment and the configuration according to the third embodiment may be combined to form a biological information measurement device including all of the blood pressure measurement unit 200, the electrocardiographic waveform measurement unit 300, and the SpO2 measurement unit 400.

In the above embodiments, when the fact that arrhythmia has been detected during the blood pressure measurement is displayed, the display mode may be made different from the display mode at the time of notifying of another measurement error or failure of the device. Specifically, the detection of arrhythmia can be clearly identified from other errors by a difference in lighting/blinking of the display, a difference in blinking interval, a difference in display color, and the like.

In the second and third embodiments, the electrocardiographic waveform measurement unit 300 and the SpO2 measurement unit 400 are exemplified as the devices integrated with the blood pressure measurement unit 200. However, the present invention is not limited thereto, and a configuration can be adopted in which the electrocardiographic waveform and the photoelectric pulse wave acquired by a separate measuring instrument are acquired via the communication unit. In this case, the communication unit serves as the arrhythmia information acquisition unit. Furthermore, the present invention can also be applied to a wearable type, such as a wristwatch type, biological information measurement device.

In the fourth embodiment, the biological information measurement device 61 includes the blood pressure measurement unit 200 and the electrocardiographic waveform measurement unit 300. However, the technology of determining the presence or absence of arrhythmia during the blood pressure measurement using another information processing terminal is naturally applicable to other biological information measurement devices as described in the first and third embodiments. Furthermore, the information processing terminal is not limited to the smartphone 62, and other computers can be adopted regardless of whether the computer is a portable computer or a stationary computer.

In each of the above examples, the description has been made using the atrial fibrillation as an example of arrhythmia. However, as a matter of course, also in a case where another arrhythmia (atrial flutter, premature contraction, etc.) has been detected, the fact that atrial flutter or premature contraction has been detected may be displayed without displaying the blood pressure value similarly to the above embodiments.

What is claimed is:

1. A biological information measurement device comprising:
a blood pressure measurement unit that acquires information for measuring blood pressure of a living body;
an arrhythmia information acquisition unit that acquires information related to presence or absence of arrhythmia in the living body during measurement of the blood pressure;
a display unit including an area capable of displaying at least the blood pressure;
an input unit that receives an operation input by a user; and
a control unit, wherein
the control unit
calculates the blood pressure on the basis of the information acquired by the blood pressure measurement unit, and
causes, in a case where the information acquired by the arrhythmia information acquisition unit indicates that arrhythmia during the measurement of the blood pressure has been present, the display unit not to display a value of the blood pressure measured but to display information of a fact that arrhythmia has been present.

2. The biological information measurement device according to claim 1, wherein
the control unit
calculates a first average value that is an average value of the blood pressure of a case where the measurement of the blood pressure is performed a plurality of times at predetermined intervals according to an operation by a user, and causes the display unit to display the first average value, and
causes, in a case where the information acquired by the arrhythmia information acquisition unit indicates that arrhythmia has been present during the measurement in any of the plurality of times of measurement of the blood pressure, the display unit not to display the first average value but to display information of the fact that arrhythmia has been present.

3. The biological information measurement device according to claim 1, further comprising
a storage unit that holds information related to measurement of the blood pressure performed in the past, the information including information related to a date and time of the measurement and presence or absence of arrhythmia during the measurement at the date and time, wherein
the control unit
receives, via the input unit, a request to display, on the display unit, the information related to the measurement performed in a past of the blood pressure, the information being held in the storage unit, and
causes, in a case where the information related to the measurement of the blood pressure related to the request to display includes information of the fact that arrhythmia has been present during the measurement, the display unit not to display a value of the blood pressure at the time of the measurement performed in a past but to display the information of the fact that arrhythmia has been present.

4. The biological information measurement device according to claim 3, wherein
the control unit
receives, via the input unit, a request to calculate a second average value that is an average value of the blood pressure for each measurement time zone in past any period using the information related to the measurement of the blood pressure held in the storage unit,
calculates, in a case where any of a plurality of pieces of the information related to the measurement of the blood pressure related to the request to calculate includes information of the fact that arrhythmia has been present during the measurement of the blood pressure, the second average value without using the information related to the measurement of the blood pressure, and
causes the display unit to display the second average value calculated.

5. The biological information measurement device according to claim 1, wherein
the control unit
detects body motion during the measurement of the blood pressure on the basis of the information acquired by the blood pressure measurement unit, and
causes, in a case where the number of times of detection of the body motion during the measurement of the blood pressure is equal to or more than a predetermined value, the display unit to display that the number of times of detection of the body motion during the measurement of the blood pressure is equal to or more than the predetermined value.

6. The biological information measurement device according to claim 1, wherein
the arrhythmia information acquisition unit acquires the information related to presence or absence of arrhythmia in the living body on the basis of pulse wave information acquired by the blood pressure measurement unit.

7. The biological information measurement device according to claim 1, further comprising
an electrocardiographic waveform acquisition unit that acquires an electrocardiographic waveform of the living body, wherein
the arrhythmia information acquisition unit acquires the information related to presence or absence of arrhythmia in the living body on the basis of the electrocardiographic waveform during the measurement of the blood pressure.

8. The biological information measurement device according to claim 1, wherein
the control unit
causes, in a case where a user operation to display a blood pressure value measured when arrhythmia has been present is received via the input unit after causing the display unit not to display the value of the blood pressure measured but to display information of the fact that arrhythmia has been present, the display unit to display the blood pressure value.

9. A control method of a biological information measurement device, comprising:
measuring blood pressure of a living body;
determining presence or absence of arrhythmia in the living body during measurement of the blood pressure; and
displaying a measurement result of the blood pressure, wherein,
in a case where it is determined, in the determining presence or absence of arrhythmia, that arrhythmia has been present, a value of the blood pressure measured is not displayed but information of a fact that arrhythmia has been present is displayed in the displaying the measurement result.

10. A non-transitory computer-readable storage medium recording a program for causing a biological information measurement device to execute the measuring, the determining, and the displaying according to claim 9.

11. An information processing system comprising:
a biological information measurement device that measures at least blood pressure of a living body; and an information processing terminal communicatively connected to the biological information measurement device, wherein
the information processing terminal
receives, from the biological information measurement device, at least determination information for determining presence or absence of arrhythmia during measurement of the blood pressure of the living body,
determines presence or absence of arrhythmia during the measurement of the blood pressure of the living body on the basis of the determination information, and
transmits, in a case where it is determined that arrhythmia has been present during the measurement of the blood pressure of the living body, information of a fact that arrhythmia has been present during the measurement of the blood pressure of the living body to the biological information measurement device, and wherein
the biological information measurement device includes a first display unit, and
causes, in a case where the information of the fact that arrhythmia has been present during the measurement of the blood pressure of the living body is received from the information processing terminal, the first display unit not to display a value of the blood pressure measured but to display the information of the fact that arrhythmia has been present.

12. The information processing system according to claim 11, wherein
the biological information measurement device includes an electrocardiographic waveform acquisition unit that acquires an electrocardiographic waveform of the living body, and
the determination information is the electrocardiographic waveform.

13. The information processing system according to claim 11, wherein
the information processing terminal includes a second display unit,
receives a blood pressure value of the living body measured by the biological information measurement device,
displays the blood pressure value received on the second display unit in a case where it is determined that arrhythmia has been absent during the measurement of the blood pressure of the living body, and does not display the blood pressure value but displays the information of the fact that arrhythmia has been present on the second display unit in a case where it is determined that arrhythmia has been present during the measurement of the blood pressure of the living body.

14. The information processing system according to claim 11, wherein
the biological information measurement device transmits pulse wave information related to the measurement of the blood pressure to the information processing terminal, and
the information processing terminal includes
a second display unit,
calculates a blood pressure value of the living body on the basis of the pulse wave information received from the biological information measurement device,
displays the blood pressure value measured on the second display unit in a case where it is determined that arrhythmia has been absent during the measurement of the blood pressure of the living body, and does not display the blood pressure value but displays the information of the fact that arrhythmia has been present on the second display unit in a case where it is determined that arrhythmia has been present during the measurement of the blood pressure of the living body.

15. The information processing system according to claim 11, wherein
the information processing terminal
includes a second display unit,
receives a blood pressure value of the living body measured by the biological information measurement device,
displays the blood pressure value received on the second display unit in a case where it is determined that arrhythmia has been absent during the measurement of the blood pressure of the living body, and does not display the blood pressure value but displays the information of the fact that arrhythmia has been present on the second display unit in a case where it is determined that arrhythmia has been present during the measurement of the blood pressure of the living body, and
displays, in a case where a user operation to display the blood pressure value measured when the arrhythmia has been present is received after causing the second display unit to display the information of the fact that arrhythmia has been present, the blood pressure value on the second display unit.

16. A non-transitory computer-readable storage medium recording a program for causing an information processing device to function as the information processing terminal according to claim 11.
